(19) United States
(12) Patent Application Publication
(10) Pub. No.: US 2010/0086595 A1
(43) Pub. Date: Apr. 8, 2010

(54) NON-IONIZABLE HYDROPHOBIC GALENICAL SYSTEM

(75) Inventors: Rosanne Raynal, Saint Orens De Gameville (FR); Nelly Lassu, Arbus (FR); Christophe Brun-Baronnat, Beaupuy (FR); Karim Ioualalen, Saint Orens De Gameville (FR)

Correspondence Address: Baker Donelson Bearman, Caldwell & Berkowitz, PC, 555 Eleventh Street, NW, Sixth Floor, Washington, DC, 20004, US

(73) Assignee: ORALANCE PHARMA FACULTE DE MEDECINE COCHIN, PARIS (FR)

(21) Appl. No.: 12/532,164

(22) PCT Filed: Mar. 21, 2008
(86) PCT No.: PCT/EP2008/053451

§ 371 Date: Sep. 20, 2009

(30) Foreign Application Priority Data
Mar. 21, 2007 (FR) 07 53964

(51) Int. Cl. A61K 9/14

(52) U.S. Cl. 424/484

(57) ABSTRACT

The present invention concerns a novel hydrophobic galenic system allowing improved masking of the taste of the active ingredients it contains, the stability of said active ingredients and, when applicable, sustained release thereof. The galenic system of the invention consists of lipid particles with no surfactants or emulsifiers, comprising a lipid hydrophobic matrix that is non-ionizable at physiological pH in which the active ingredient(s) are dispersed. This system is suitable for the preparation of pharmaceutical or veterinary compositions, in particular for administration via oral route or via injection.

The present invention concerns a novel hydrophobic galenic system allowing improved masking of the taste of the active ingredients it contains, the stability of said active ingredients and, when applicable, their sustained release. The galenic system of the invention is suitable for the preparation of pharmaceutical or veterinary preparations, in particular for administration via oral route or via injection.

### BACKGROUND OF THE INVENTION

The chief difficulties encountered with the oral administering of an active ingredient vary in relation to their presentation.

For dry forms such as tablets and hard and soft capsules, the disadvantages are swallowing and taste. Some populations such as elderly persons, children and certain persons with mental disorders need to turn to facilitated forms of oral administration.

With regard to dispersed and liquid forms, these are easy to take but this form always come up against a non-solved problem of taste-masking and the instability of numerous active ingredients in an aqueous phase.

Finally, there are also problems of irritability, mucosal toxicity and gastro-toxicity which are encountered when taking certain medications such as anti-inflammatories.

One solution to obtain effective masking is the incorporation of the active ingredient into hydrophobic particles such as described in patent applications WO 99/65448, WO 2004/084856 and WO 2006/070117. However, these efficient compositions require the use of mineral fillers, fatty acids or agents neutralizing the incorporated active ingredient.

We have now unexpectedly shown that the efficacy of taste-masking can be further improved by using hydrophobic compositions which are not ionizable in physiological fluids and which do not contain mineral fillers.

### BRIEF DESCRIPTION OF THE INVENTION

The present invention therefore concerns a galenic system in the form of lipid particles without any surfactants or emulsifiers and containing at least one active ingredient, characterized in that the particles consist of a lipid, hydrophobic matrix that is non-ionizable at physiological pH, in which the active ingredient(s) are dispersed so that the surface of the lipid particles is devoid of active ingredient.

The invention also concerns a process to mask the taste of active ingredients and/or to protect the active ingredients against degradation reactions and/or to modulate the rate of release of the active ingredients, characterized in that lipid particles are prepared without any surfactants or emulsifiers and comprise a lipid, hydrophobic matrix non-ionizable at physiological pH in which the active ingredient(s) are dispersed.

The matrix may advantageously be called “strictly hydrophobic” insofar as it does not contain detectable traces of water.

The hydrophobic matrix is non-ionizable if its constituent elements are not able to be ionized at physiological pH, in particular do not contain any acid or basic functions, such as organic or mineral acids and organic or mineral bases. More particularly, the particles of the galenic system according to the invention do not contain any fatty acids.

The lipid, hydrophobic matrix is advantageously chosen from among natural or synthetic oils or waxes that cannot be ionized at physiological pH, and their mixtures and in particular triglycerides and derivatives, palm oil, Carnauba wax, Candellila wax, Alfa wax, cocoa butter, vegetable waxes, rice wax, hydrogenated jojoba wax or florali absolute waxes, beeswaxes and modified beeswaxes, fatty alcohols, esters of fatty acids and of alcohols with high molecular weight, sterols such as cholesterol and its esters, vegetable oils such as olive oil, groundnut oil, oils of hydrophobic silicones, cyclomethicones, vaseline, paraffin oil, paraffin, linear alkanes and their mixtures.

Preferably, the chosen constituents do not have any polymorphism properties.

The composition is more particularly devoid of any traces of water or organic solvents. It is strictly hydrophobic.

The active ingredient or ingredients are dispersed in the lipid matrix, and are essentially distributed with constant concentration in said matrix. Depending upon the hydrophilic or hydrophobic nature of the active ingredient, it is solubilized in full or in part in the hydrophobic matrix or is simply dispersed.

The particles advantageously have an active ingredient filling ratio of between 0.10 and 2 grams/gram of hydrophobic lipid matrix, more preferably between 0.5 and 2 grams/gram of hydrophobic lipid matrix, and further preferably between 1 and 2 grams/gram of hydrophobic lipid matrix.

They preferably have a melting point of between 15 and 85° C., more preferably between 30 and 45° C.

In best preferred manner, the particles are spherical.

Their size advantageously lies between 0.5 and 1500 μm, preferably between 100 and 400 μm for administration via oral route, preferably between 0.5 and 5 μm for administration via injection.

The invention also concerns a pharmaceutical or veterinary composition in the form of an oral powder, pill, tablet, hard capsule, soft capsule, gum, oral liquid, drops, syrup, suspension, solution, injectable or implantable solution or suspension, and containing particles according to the invention.

### DETAILED DESCRIPTION OF THE INVENTION

The compositions conforming to the invention essentially consist of waxes or wax mixtures, whether vegetable, animal or mineral, natural, synthetic or hemisynthetic, of non-amphiphilic oils and of hydrophobic compounds allowing adjustment of melting point and of physicochemical properties such as hardness. They may further contain oily, pasty or solid additives, fat-soluble or water-soluble active ingredients. Mixtures may be used whose melting point lies between 15° C. and 85° C. In one particular embodiment of the invention, a hydrophobic composition is used whose melting point, after incorporation of the active ingredient, lies between 15 and 50° C., more preferably between 30 and 45° C. A suitable composition is to be chosen, appropriate for absorption via oral route or any other administration mode.

The components are preferably chosen from among components already used for administering via oral route and described in the list of excipients published by the European Pharmacopeia or in the GRAS list published by the FDA, so that the formed particles maintain their incorporating, taste-masking and active ingredient stabilizing properties.

The composition contains waxes or wax mixtures preferably chosen from among:
glycerides, hemisynthetic glycerides, and derivatives with long or medium length chains, able to be hydrogenated,
palm oil, sesame oil, Carnauba wax oil,
Candellila wax, Alfa wax, Carnauba wax,
cocoa butter
ozokerite
vegetable waxes such as olive wax, rice wax, hydrogenated jojoba wax or florali absolute waxes,
beeswaxes and modified beeswaxes.

It is possible to use other compounds, such as fatty alcohols with high molecular weight e.g. cetanol, myristoyl alcohol, stearoyl alcohol, the esters of acids and alcohols with high molecular weight in particular the esters of linear and saturated acids with even carbon numbers C14 to C20 and linear and saturated alcohols with even carbon numbers C14 to C32. In all cases, the mixture obtained must be characterized by an end melting point of between 15° C. and 85° C., by the absence of surfactant compounds, by hydrophobic behaviour and by non-wettability with water. In addition to the above-mentioned waxes, the composition of the invention may contain an oil or a mixture chosen from among:
oils of hydrophobic silicones with a viscosity of between 5 and 9,000 centistokes, cyclomethicones
lipophilic organic fluorinated oils,
perhydrosqualene.

Other oily compounds may be used such as oleic alcohol, lanoline, sunflower oil, palm oil, olive oil, but the mixture obtained must be characterized by hydrophobic behaviour, by lack of miscibility with water, and by a melting point of between 15° C. and 85° C.

It is additionally possible, in order to adjust consistency, to add clays or their oily dispersions to the composition, or gums of phenylated silicones, starches, fat structuring agents.

The person skilled in the art will know how to choose the suitable waxes, oils, and additives to obtain the desired properties of the particles according to the invention, and in particular the absence of compounds ionizable at physiological pH such as fatty acids and surfactants.

According to one particular embodiment of the invention, the hydrophobic matrix solely consists of waxes non-ionizable at physiological pH.

The matrix of the invention is advantageously strictly hydrophobic.

The preparation of the particles according to the invention is well known to the person skilled in the art, in particular by following the operating modes described in patent applications WO 99/65448, WO 2004/084856 and WO 2006/070117.

The particles of the invention are obtained by mixing under moderate heat. More precisely, these compositions are obtained using a process characterized by the fact that the wax and oil are mixed at the temperature of the melting point of the wax, until a mixture is obtained characterized by a melting point lower than the melting point of the wax. The initial ratio between the wax and the oil can be modulated so that the melting point of the end mixture is lower than the degradation temperature of the most heat-sensitive compound to be incorporated. The end mixture must be solid at the temperature of utilization and in one of its preferred forms must have a melting point of 37.5° C. The mixture is then cooled under adapted stirring, at a temperature 2° C. or 3° C. above its melting point to allow the addition of the active pharmaceutical ingredients. The mixture is then shaped to give hydrophobic spherical particles called particles.

To the hydrophobic matrix consisting of the waxes, oils and mixtures, it is possible to add a certain number of compounds such as mineral fillers allowing modulation of density and plasticity. Amongst the mineral compounds, talc and kaolin are advantageously chosen.

According to another particular embodiment of the invention, the particles do not contain any mineral filler.

According to one preferred embodiment of the invention, the size of the particles ranges from 0.5 to 1,500 micrometres and they contain cosmetic, pharmaceutical, biotechnological components dissolved or dispersed in their matrix. According to this embodiment, the mixture may contain components such as essential oils, flavourings, pigments, fillers, colouring agents, enzymes and coenzymes, and other active substances. The filling ratio of the particles may extend from 0.02% to 75% relative to the weight of the particles. The person skilled in the art knows that when these components are incorporated in the particles, a suitable lipid composition must be chosen so that the process can be implemented and the particles are solid at the temperature of utilization with a size preferably ranging from 0.5 to 1,500 micrometres, and preferably from 100 to 400 microns for administration via oral route.

Among the components which may be incorporated in the particles, mention may be made of vitamins or provitamins A, B, C, D, E, PP and their esters, carotenoids, anti-radical substances, hydroxyacids, antiseptics, molecules acting on pigmentation, on inflammation, biological extracts. The particules may also contain preservatives, antioxidants, colouring agents and pigments, cells and cell organelles. Finally, these particles may contain pharmaceutical components intended to treat skin or mucosal pathologies.

In this description, the term active component is used to designate any active therapeutic substance or mixture able advantageously to be administered to man or to animals to diagnose, care for, reduce, treat or prevent disease. As examples, mention may be made of antibiotics, macrolides, antifungals, itraconazole, ketoconazole, antiparasitics, antimalarials, adsorbents, hormones and derivatives, nicotine, antihistaminics, steroid and non-steroid anti-inflammatories, ibuprofen, naproxene, cortisone and its derivatives, anti-allergy agents, analgesics, local anesthetics, antivirals, antibodies and molecules acting on the immune system, cytostaties and anticancer agents, analgesics, hypolipidemics, vasodilators, vasoconstrictors, inhibitors of angiotensin-converting enzyme and phosphodiesterase, fenofibrate and derivatives, statins, nitrate derivatives and anti-anginals, beta-blockers, calcium inhibitors, anti-diuretics and diuretics, bronchodilators, opiates and derivatives, barbiturates, benzodiazepines, molecules acting on the central nervous system, nucleic acids, peptides, anthracenic compounds, paraffin oil, polyethylene glycol, mineral salts, antispasmodics, gastric anti-secretory agents, clay gastric dressings and polyvinylpyrrolidone, aluminium salts, calcium carbonates, magnesium carbonates, starch, derivatives of benzimidazole. This exhaustive list is not at all limiting.

Preferably, the lipid particles of the invention are devoid of active ingredient on their surface. Elimination of active ingredient on the surface is obtained, for example, by washing the particles or by any other suitable method.

Since the lipid particles are devoid of active ingredient on their surface, it is not necessary to have recourse to coating the particles to mask the taste of the active ingredients and/or to protect the active ingredients against degradation reactions and/or to modulate the rate of release of the active ingredients.

The lipid particles of the invention can therefore be directly formulated as powders, compressed powders, aqueous suspensions or syrups.

To the dry powder of particles according to the invention, it is also possible to add lubricating agents which improve the fluidity of particles such as talc, starches, silica powders, antistatic agents. Evidently, the particles of the invention can be used in any adequate galenic formulation. In one advantageous form of embodiment, the particles of the invention are used in aqueous suspensions, syrups, and sachets. Finally, the particles can be used in conventional galenic formulations of the type hard capsules, soft capsules, granules, oral powders, dispersible powders, tablets, water-dispersible and oral-dispersible tablets.

The invention therefore also concerns processes to prepare powders, compressed powders, aqueous suspensions or syrups containing lipid particles according to the invention.

According to a second aspect of the invention, we have found that the taste-masked particles of the invention can also be used for administering via injection and in particular for the preparation of forms with sustained release. In this case, the particles of the invention are prepared so as to have a size of preferably between 0.5 and 5 μm. Preferably they are sieved to obtain a size distribution conforming to the administration mode. Their waxy composition is chosen to conform to the requirements of injectable route. With this galenic form, it is possible to eliminate toxicity problems encountered with polymeric particles obtained using emulsion polymerisation processes and related to the use of solvents and surfactant compounds. The particles of the invention allow filling ratios of active ingredient to be obtained of between 0.10 and 2 grams/gram of hydrophobic lipid matrix. The person skilled in the art knows that encapsulation technologies do not allow such ratios to be achieved. Finally, the degradation of the particles does not lead to an inflammatory reaction as may be encountered with injectable polymers containing a polylactic-glycol polymer.

The following examples, which are non-limiting, serve solely to illustrate the invention. Also, for some of the following examples, the taste-masking tests were conducted on a sample of ten individuals. The results are expressed using the following scale:
1: the taste of the active ingredient is not detected.
2: the taste of the active ingredient is slightly perceived.
3: the taste of the active ingredient is detected.
4: the taste of the active ingredient remains acceptable.
5: the taste of the active ingredient is not acceptable.

### EXAMPLES

### Example 1

### Particles Containing Ibuprofen

Example given for the preparation of 35 g of particles containing 10 g of ibuprofen:

Composition:

| | |
|---|---|
| Triglycerides of hydrogenated palm oil | 24 g |
| melting point 40° C. | |
| Trilaurine | 1 g |
| Ibuprofen | 10 g |

Operating Mode:

In a thermostat-controlled container, the compound with the highest melting point is brought to 2° C. above its melting point, then the different compounds are gradually added starting with the compound with the next highest melting point down to the compound with the lowest melting point. The temperature of the mixture is gradually lowered so that it is held 2° C. to 3° C. above the melting point of the new mixture obtained. The ibuprofen is added last. The dispersion of these compounds in the lipid phase is achieved using a stirring system equipped with an anchor-shaped impeller at a speed of 400 rpm. When the mixture is homogeneous, it is added to 500 ml of 0.4% aqueous gel of Carbopol Ultrez 10 neutralized to pH 6.2 with sodium hydroxide, previously brought to the same temperature as the lipid mixture and contained in a reactor equipped with a stirring system with four-blade propeller. When adding the composition, the stir rate of the propeller is 400 rpm. Stirring is continued for 30 seconds after the completed adding of the composition, and is then stopped. The dispersion is then cooled to 15° C. The particles are collected by screening then washed in distilled water, after which they are collected and dried. The particles so obtained have a mean size of 285 microns.

The taste-masking test conducted on the particles did not give rise to any detection of the active ingredient. On the particles, and after extraction, the ibuprofen was assayed by HPLC Dionex, column: Prontosil C18 150×4.6 mm, 1 ml/mn, isocratic mode, UV detection 220 nm. Mobile phase (CH3CN/0.2% orthophosphoric acid: 60/40), tR of ibuprofen 5.8 minutes.

10 g ibuprofen are obtained per 35 g of particles.

### Example 2

### Preparation of a Syrup Containing Particles Carrying Ibuprofen

Using a pharmaceutical sucrose syrup distributed by Cooper, called Simple Syrup with the composition:

| | | |
|---|---|---|
| Sucrose | 86.5 | g |
| Sodium methyl para-hydroxybenzoate | 0.15 | g |
| Sodium propyl para-hydroxybenzoate | 0.03 | g |
| Purified water | qs 100 | ml |

To 250 ml of syrup and at room temperature, 20 g of ibuprofen-carrying particles are added as obtained in accordance with Example 1, which corresponds to 5.71 g of ibuprofen. The taste test conducted on the syrup did not give rise to any detection of the active ingredient.

### Example 3

### Preparation of a Water-Dispersible Powder for Oral Administration, Containing Ibuprofen-Carrying Particles

The following are placed in a powder mixer:

| | |
|---|---|
| Particles according to Example 1 | 100 g |
| Mannitol | 208.5 g |
| Flavouring | 1 g |
| Aspartame | 3 g |
| Xanthan gum | 0.5 g |
| Talc | 7 g |

After mixing, the whole is divided into unit sachets of 2.13 g containing 200 mg of ibuprofen. Dissolving in 50 ml of water allows reconstitution of an aqueous dispersion of the anti-inflammatory. The taste test conducted on the dispersion did not give rise to any detection of the active ingredient.

### Example 4

### Sustained-Release Particles Containing Ibuprofen

Example given for the preparation of 35 g of particles containing g of ibuprofen:

Composition:

| | |
|---|---|
| Hydrogenated triglycerides, melting point 40° C. | 17 g |
| Paraffin | 6 g |
| Trilaurine | 1 g |
| Tribehenate | 1 g |
| Ibuprofen | 11.3 g |

The particles are prepared following the protocol of Example n^{o} 1. On completion, 35 g of particles are finally obtained containing 10 g of ibuprofen.

### Example 5

### Particles Containing Paracetamol

Example given for the preparation of 100 g of particles enabling the taste of paracetamol to be reduced:

Composition:

| | |
|---|---|
| Triglycerides of hydrogenated palm oil | 40 g |
| melting point 42° C. | |
| Triglycerides of hydrogenated palm oil | 32 g |
| melting point 38° C. | |
| Talc | 2 g |
| Tribehenate | 1 g |
| Paracetamol | 25 g |

The operating mode is identical to the one described in Example 1.

The taste-masking test conducted on the particles did not give rise to any detection of active ingredient.

### Example 6

### Particles Containing Copper Sulphate and Calcium Ascorbate

Example given for the preparation of 70 g of particles containing calcium ascorbate and copper sulphate:

Composition:

| | | |
|---|---|---|
| Triglycerides of hydrogenated palm oil | 55 | g |
| melting point 42° C. | | |
| Tribehenate | 7 | g |
| Carnauba wax | 2.8 | g |
| Calcium ascorbate | 4 | g |
| Copper sulphate | 120 | mg |

The operating mode is identical to the one described in Example 1.

The taste-masking test performed on the particles did not give rise to any detection.

### Example 7

### Particles Containing Clarythromycine

Example given for the preparation of 100 g of particles containing clarythromycine:

Composition:

| | |
|---|---|
| Hydrogenated triglycerides | 60 g |
| melting point 42° C. | |
| Hydrogenated palm oil | 10 g |
| Clarythromycine | 30 g |

The operating mode is identical to the one described in Example 1.

The taste-masking test performed on the particles did not give rise to any detection.

1. A process to mask the taste of active ingredients and/or to protect active ingredients against degradation reactions and/or to modulate the rate of release of active ingredients, wherein lipid particles are prepared without any surfactants or emulsifiers comprising a lipid hydrophobic matrix that is non-ionizable at physiological pH, in which the active ingredient(s) are dispersed such that the surface of the lipid particles is devoid of active ingredient.

2. A process according to claim 1, wherein the lipid hydrophobic matrix is chosen from among natural or synthetic oils or waxes that are non-ionizable at physiological pH, and/or their mixtures thereof.

3. A process according to claim 1, wherein the hydrophobic matrix comprises waxes which are non-ionizable at physiological pH.

4. A process according to claim 1, wherein the particles do not contain any mineral fillers.

5. A process according to claim 1, wherein the constituents of the lipid hydrophobic matrix are chosen from among triglycerides and derivatives, palm oil, Carnauba wax, Candellila wax, Alfa wax, cocoa butter, vegetable waxes, rice wax, hydrogenated jojoba wax or florali wax absolutes, beeswaxes and modified beeswaxes, fatty alcohols, the esters of fatty acids and alcohols with high molecular weight, sterols such as vegetable oils, groundnut oil, oils of hydrophobic silicones, cyclomethicones, vaseline, paraffin oil, paraffin, linear alkanes and/or mixtures thereof.

6. A process according to claim 1, wherein the composition is devoid of traces of water or of organic solvents.

7. A process according to claim 1, wherein the lipid hydrophobic matrix has a melting point of from 15° C. to 85° C.

8. A process according to claim 1, wherein the particles have a filling ratio of active ingredient of between 0.10 and 2 grams/gram of hydrophobic lipid matrix.

9. A process according to claim 1, wherein the particles comprise at least one additive chosen from among essential oils, flavourings, pigments, fillers, colouring agents, enzymes, and coenzymes and/or mixtures thereof.

10. A process according to claim 1, wherein the particles have a melting point of from 15 to 50° C.

11. A process according to claim 1, wherein the particles are spherical.

12. A process according to claim 1, wherein the size of the particles lies from 0.5 to 1,500 μm.

13. A process according to claim 12, wherein the size of the particles lies from 100 to 400 μm

14. A process according to claim 12, wherein the particles have a size of from 0.5 to 5 μm.

15. A process according to claim 1, wherein the particles are then formulated in a pharmaceutical or veterinary composition, optionally in the form of an oral powder, pill, tablet, hard capsule, soft capsule, gum, oral liquid, drops, syrup, suspension, solution, and/or injectable solution.

16. A pharmaceutical and/or veterinary composition for administration via an oral route, wherein said composition comprises particles prepared by a process of claim 15.

17. A pharmaceutical and/or veterinary composition for administration via an injection route, wherein said composition comprises particles prepared by a method defined in claim 15.

18. A galenic system in the form of lipid particles without any surfactants or emulsifiers comprising at least one active ingredient, wherein the particles comprise a lipid hydrophobic matrix that is non-ionizable at physiological pH in which the active ingredient(s) are dispersed, the hydrophobic matrix comprising non-ionizable hydrophobic waxes.

19. A galenic system according to claim 18, wherein the particles have a size from 0.5 to 5 μm.
